(19) United States
(12) Patent Application Publication
(10) Pub. No.: US 2014/0377318 A1
(43) Pub. Date: Dec. 25, 2014

(54) ANTIMICROBIAL SANITIZER SYSTEMS AND METHODS OF KILLING OR INHIBITING PATHOGENS

(71) Applicant: S. Douglas Cornell, Tucson, AZ (US)

(72) Inventor: S. Douglas Cornell, Tucson, AZ (US)

(21) Appl. No.: 14/482,254

(22) Filed: Sep. 10, 2014

Related U.S. Application Data
(63) Continuation-in-part of application No. PCT/US13/73318, filed on Dec. 5, 2013.
Application No. PCT/US13/73318 is a continuation-in-part of application No. 13/707,142, filed on Dec. 6, 2012, now Pat. No. 8,858,515.

(51) Int. Cl. A61L 31/08; A61L 31/16; A61K 33/38; A61K 33/34; A61K 33/24; A61K 33/30

(52) U.S. Cl. CPC A61L 31/088; A61K 33/24; A61K 33/30; A61K 33/38; A61K 33/34; A61L 31/16; A61L 2420/04
USPC 424/411; 424/630; 424/641; 424/618; 424/649

(57) ABSTRACT

Methods and systems for killing or inhibiting pathogens present in treatments area including but not limited to the nasal cavity, the vaginal area, and the anal area. The methods feature introducing a substrate with an antimicrobial metal into the treatment area. The substrate may be coated, lined, plated, or clad with the antimicrobial metal, either partially or entirely. Or, the substrate itself may be an antimicrobial metal. Antimicrobial metals include but are not limited to copper, zinc, silver, gold, and certain metal alloys. The step of directly contacting the substrate with antimicrobial metal to the treatment area kills or inhibits present pathogens. The antimicrobial metal may leave a trace amount of residue, which may continue to kill or inhibit pathogens after the system has been removed from the treatment area or penetrate into deeper layers of the treatment area.

### CROSS REFERENCE

This application is a continuation-in-part and claims priority to U.S. patent application Ser. No. 13/707,142, filed Dec. 6, 2012 and claims priority to International Application No. PCT/US13/73318 filed Dec. 5, 2013, the specification(s) of which is/are incorporated herein in their entirety by reference.

### FIELD OF THE INVENTION

The present invention relates to methods for killing or inhibiting pathogens, for example using antimicrobial sanitizer systems for swabbing/contacting mucous membranes, mucous, epithelial membranes, skin, vaginal tissue, vaginal secretions, anal areas (e.g., anus, rectum, lower colon, etc.), etc.

### BACKGROUND OF THE INVENTION

The present invention features methods and systems for killing or inhibiting pathogens. The pathogens (e.g., pathogens associated with colds, the flu, methicillin-resistant Staphylococcus aureus (MRSA), etc.) may be present in the mucous or mucous membranes of the nasal cavity. The pathogens may be present in other locations. For example, pathogens (e.g., pathogens associated with sexually transmitted illnesses like herpes simplex virus (HSV), human papillomavirus (HPV), human immunodeficiency virus (HIV), etc.) may be present in the vaginal tissue or secretions, or in an anal area (e.g., anus, rectum, etc.). The methods feature introducing a system, e.g., a substrate comprising an antimicrobial metal, to the area to be treated and using the system to directly contact the pathogens residing in the treatment area (e.g., mucosal area, vaginal area, anal area, skin, etc.). The direct contact of the antimicrobial metal causes the killing or inhibition of the pathogen. The antimicrobial metal may leave a trace amount of residue, which may continue to kill or inhibit pathogens after the system has been removed from the treatment area.

The methods and systems of the present invention may be used to treat or prevent various conditions associated with various pathogens. For example, the methods and systems, when applied to the mucosal areas, may be used to treat or prevent colds, the flu, MRSA, or other illnesses transmitted through the nose. Or, the methods and systems, when applied to vaginal or anal areas, may be used to treat or prevent sexually transmitted illnesses such as HSV, HPV, etc.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features a method of killing or inhibiting pathogens of a treatment area, e.g., a mucosal area (e.g., mucosal membrane, mucous in a nasal cavity, etc.). In some embodiments, the method comprises locating a mucosal area; introducing a substrate to the mucosal area, the substrate comprising an antimicrobial metal; and using the substrate to directly contact the pathogens residing in the mucosal area, wherein the method is intended to kill or inhibit the pathogens in the mucosal area.

The present invention also features a method of killing or inhibiting pathogens of a treatment area, e.g., a vaginal area (e.g., vaginal secretions, vaginal wall, cervical tissue, etc.). In some embodiments, the method comprises locating a vaginal area; introducing a substrate to the vaginal area, the substrate comprising an antimicrobial metal; and using the substrate to directly contact the pathogens residing in the vaginal area, wherein the method is intended to kill or inhibit the pathogens in the vaginal area.

The present invention also features a method of killing or inhibiting pathogens of a treatment area, e.g., an anal area (e.g., anus, rectum, lower colon, etc.). In some embodiments, the method comprises locating an anal area; introducing a substrate to the anal area, the substrate comprising an antimicrobial metal; and using the substrate to directly contact the pathogens residing in the anal area, wherein the method is intended to kill or inhibit the pathogens in the anal area.

In some embodiments, the substrate comprises a non-microbial metal material either fully or partially coated with the antimicrobial metal. In some embodiments, the non-microbial metal material comprises plastic. In some embodiments, the antimicrobial metal is coated, lined, clad, or plated onto the substrate. In some embodiments, the substrate comprises a combination of antimicrobial metal and non-antimicrobial metal. In some embodiments, the substrate comprises only antimicrobial metal.

In some embodiments, the antimicrobial metal comprises copper, zinc, silver, gold, an antimicrobial alloy, or a combination thereof. In some embodiments, the antimicrobial alloy comprises brass, bronze, or a combination thereof.

In some embodiments, the substrate comprises a coil. In some embodiments, the substrate comprises a device adapted to be applied to the mucosal area. In some embodiments, the device comprises a handle, the substrate being attached to the handle. In some embodiments, the step of directly contacting the pathogens residing in the mucosal area comprises sliding the substrate around an inner cavity of the nostril to contact the mucous or mucous membranes with the substrate. In some embodiments, the step of directly contacting the pathogens residing in the mucosal area with the substrate deposits a trace amount of antimicrobial metal ions onto the mucosal area. In some embodiments, the trace amount of antimicrobial metal helps to kill or inhibit the pathogens.

In some embodiments, the substrate comprises a device adapted to be applied to the vaginal area. In some embodiments, the step of directly contacting the pathogens residing in the vaginal area with the substrate comprises sliding the substrate around the vaginal area to contact the tissues with the substrate. In some embodiments, the step of directly contacting the pathogens residing in the vaginal area with the substrate deposits a trace amount of antimicrobial metal ions onto the vaginal area. In some embodiments, the trace amount of antimicrobial metal helps to kill or inhibit the pathogens.

In some embodiments, the substrate comprises a device adapted to be applied to an anal area. In some embodiments, the step of directly contacting the pathogens residing in the anal area with the substrate comprises sliding the substrate around the anal area to contact the tissues with the substrate. In some embodiments, the step of directly contacting the pathogens residing in the anal area with the substrate deposits a trace amount of antimicrobial metal ions onto the anal area. In some embodiments, the trace amount of antimicrobial metal helps to kill or inhibit the pathogens.

### BRIEF DESCRIPTION OF THE DRAWINGS

FIG. 1 is a perspective view of an embodiment of a system of the present invention.

FIG. 2 is a front view of an embodiment of a system of the present invention.

FIG. 3A is a side view of an embodiment of a system of the present invention. An arc shape of the stem and the base handle is shown.

FIG. 3B is a side view of an embodiment of a system of the present invention. An arc shape of the stem and the base handle is shown.

FIG. 4 is a perspective view of an embodiment of a system of the present invention. The stem is optionally retractable.

FIG. 5 is a front view of an embodiment of a system of the present invention. The stem is optionally retractable.

FIG. 6 is a side and partial cross-sectional view of an embodiment of the system of the present invention. The stem is optionally retractable.

FIG. 7 is a front view of an embodiment of a system of the present invention. The stem is optionally retractable.

FIG. 8 is a perspective view of an embodiment of the system of the present invention.

FIG. 9 is a perspective view of an embodiment of the system of the present invention.

FIG. 10 is a top view of an embodiment of the system of the present invention.

FIG. 11 is a side view of an embodiment of the system of the present invention. A base handle angle is shown.

FIG. 12 is a bottom view of an embodiment of the system of the present invention.

FIG. 13 is a front view of an embodiment of the system of the present invention.

FIG. 14 is a close-up of a top view of an embodiment of the system of the present invention.

FIG. 15 is a close-up of a top view of an embodiment of the system of the present invention.

FIG. 16 is a close-up of a side view of an embodiment of the system of the present invention.

FIG. 17 is a close-up of a side view of an embodiment of the system of the present invention.

FIG. 18 is a view of an embodiment of the system of the present invention held in a hand.

FIG. 19 is a view of an embodiment of the system of the present invention held in a hand.

FIG. 20 is a view of an embodiment of the system of the present invention held in a hand.

FIG. 21 is a view of an embodiment of the system of the present invention in use.

FIG. 22 is a view of an embodiment of the system of the present invention in use.

FIG. 23 is a view of an embodiment of the system of the present invention in use.

FIG. 24 is a view of an embodiment of the system of the present invention in use.

FIG. 25 is a view of an embodiment of the system of the present invention in use.

FIG. 26 is a side view of an embodiment of the system of the present invention.

### DESCRIPTION OF PREFERRED EMBODIMENTS

Following is a list of elements corresponding to a particular element referred to herein:
100 Antimicrobial system
110 Base handle
111 Stem end
112 Bottom end
114 Front surface
115 Back surface or bottom surface
116 Inner cavity
117 Opening or aperture
118 Concave indention
119 Stopper
120 Stem
121 Distal end
121a Ridge or protrusion
122 Proximal end
130 Flange
138 Inward curvature
140 Hole or cavity
140a First cavity
140b Second cavity
148a First cavity protrusion
148b Second cavity protrusion
152 Tongue
154 Friction nub
162 Slit
180 Retention tab
200 Base mid-section
202 Base anterior handle component
204 Base posterior handle component
206 Base first side
208 Base second side
210 Base handle angle
212 Base anterior taper
214 Base posterior taper
216 Base mid-section width
218 Base stem end width
220 Base bottom end width
230 Thumb indention
232 Thumb indention depth
240 Fingertip indention (posterior indentation)
242 Fingertip indention depth
250 Ridge
252 Concave indention depth
253 First concave indention depth
254 Second concave indention depth
255 Third concave indention depth
260 Distal end width
262 Distal end thickness
264 Distal end first side
265 Distal end second side
266 Distal end top side
267 Distal end bottom side
270 Stem width
272 Stem thickness
500 Antimicrobial system
510 handle
520 stem (e.g., substrate)

### Methods

The present invention features methods of killing or inhibiting pathogens. The pathogens may be present in any appropriate treatment area. For example, in some embodiments, the pathogens may be present in a mucosal area. A mucosal area includes (and is not limited to) nasal membranes or surfaces, mucous or other nasal secretions, the space in the nasal cavity, or any other nasal cavity-related surface or secretion. In some embodiments, the pathogens may be present in the vaginal area. The vaginal area includes (and is not limited to) vaginal wall surfaces, vaginal secretions, cervical tissue, or any other vaginal- or cervical-related surface or secretion. The treatment area is not limited to a mucosal area or a vaginal area. For example, in some embodiments, the pathogens may be present in the oral cavity, the ear canal, a skin surface, the anal area (e.g., rectum, lower colon), or the like.

The method further comprises introducing a substrate to the treatment area (e.g., mucosal area, vaginal area, anal area, etc.). The substrate comprises an antimicrobial metal adapted to kill or inhibit the pathogens in the treatment area. Antimicrobial metals may include but are not limited to copper, zinc, silver, gold, an appropriate antimicrobial metal alloy (e.g., brass, bronze, etc.), any other appropriate antimicrobial metal or alloy, or a combination thereof.

The substrate may be constructed in a variety of different configurations and from a variety of different materials. The substrate itself may be an antimicrobial metal (e.g., as described above). In some embodiments, the substrate is a non-antimicrobial metal material (e.g., plastic, etc.) and is coated (e.g., fully or partially) with an antimicrobial metal. A coating may be achieved in a variety of ways, e.g., via lining, cladding (e.g., laser cladding), metal plating, or the like. In some embodiments, the substrate is a combination of an antimicrobial metal and a non-antimicrobial metal material. In some embodiments, the substrate comprises a solid piece of material, e.g., a spherical piece, a globular piece, an ellipsoidal piece, a cuboidal piece, or any other appropriately shaped piece. In some embodiments, the substrate comprises a coil.

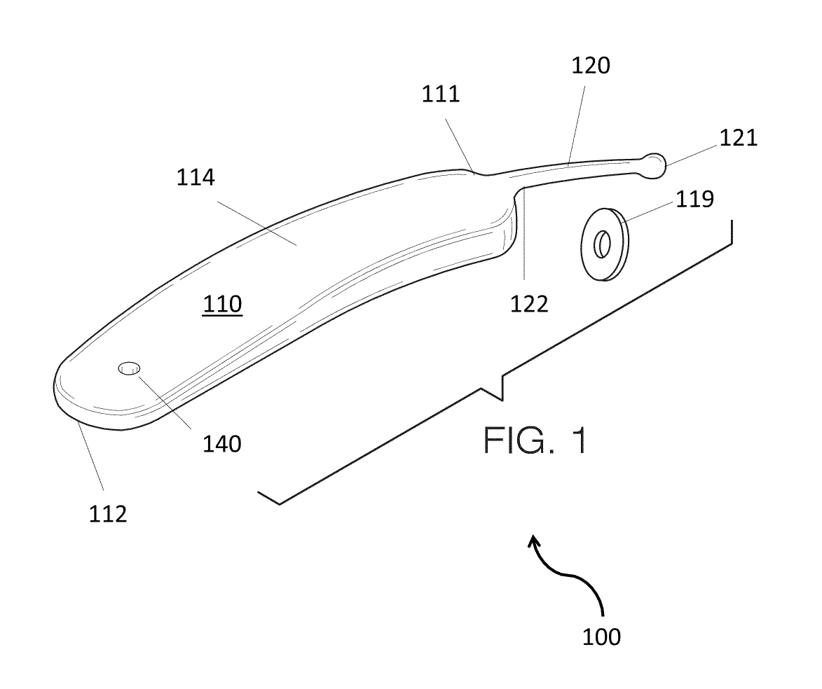

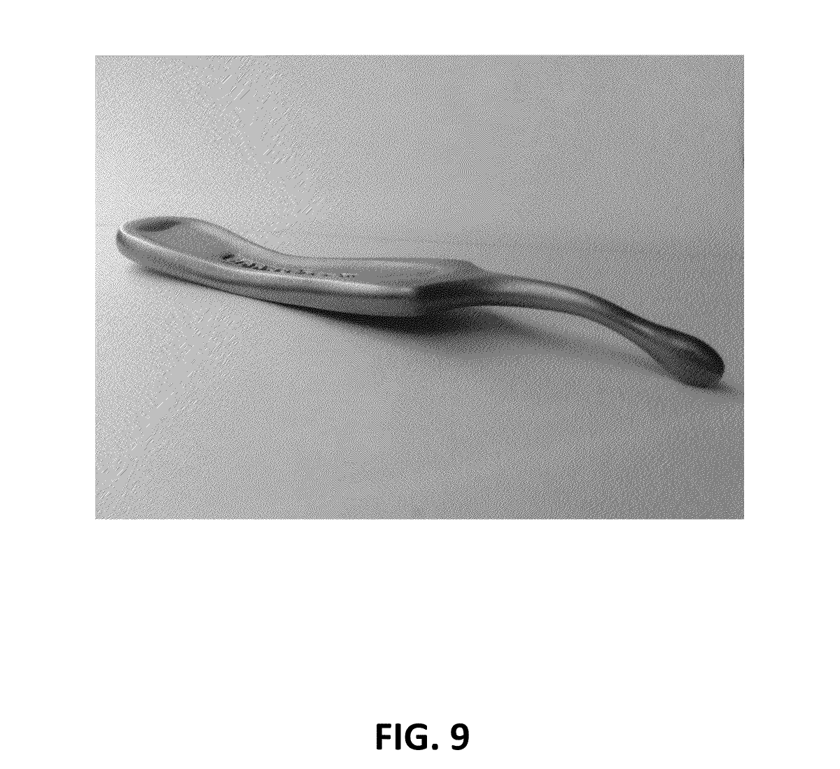

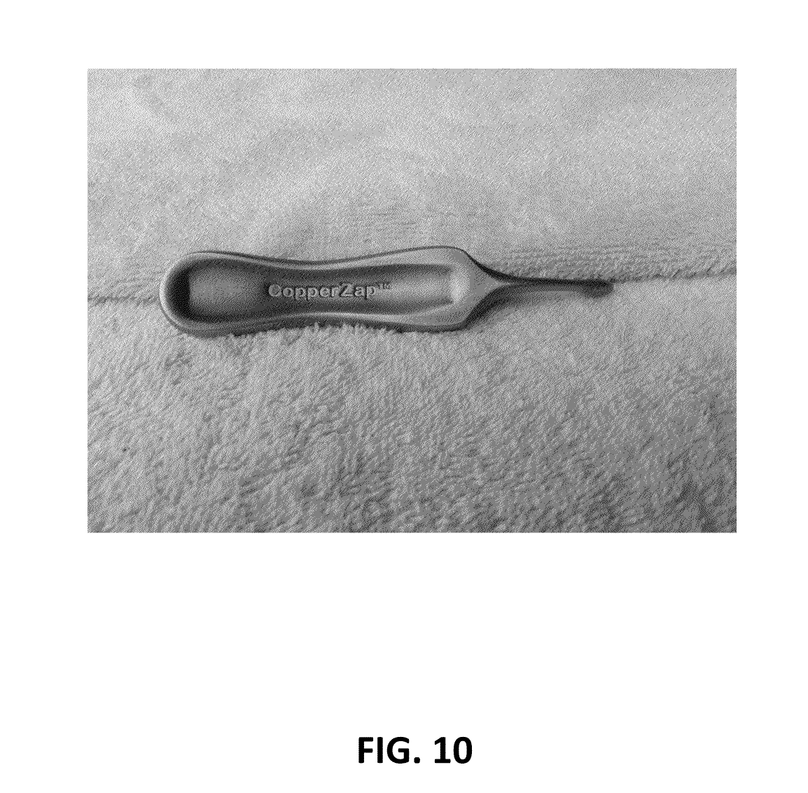

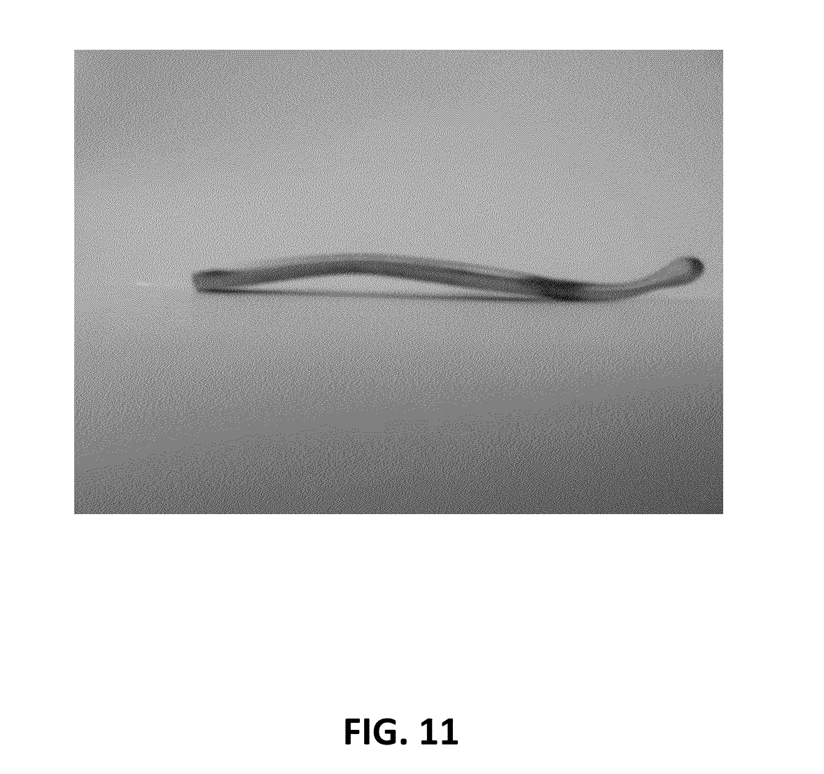

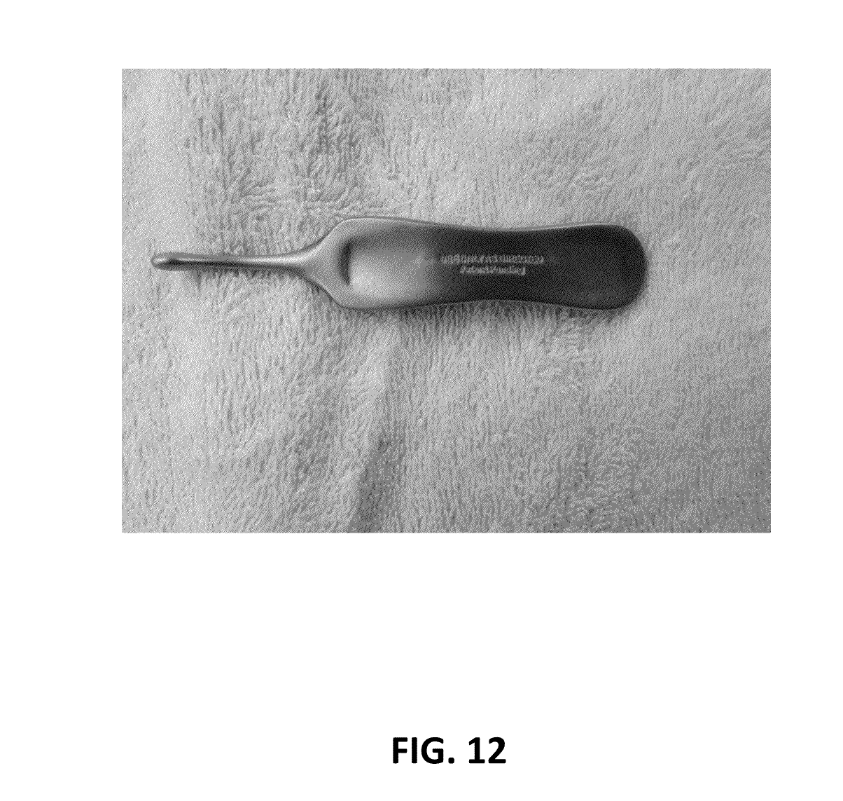

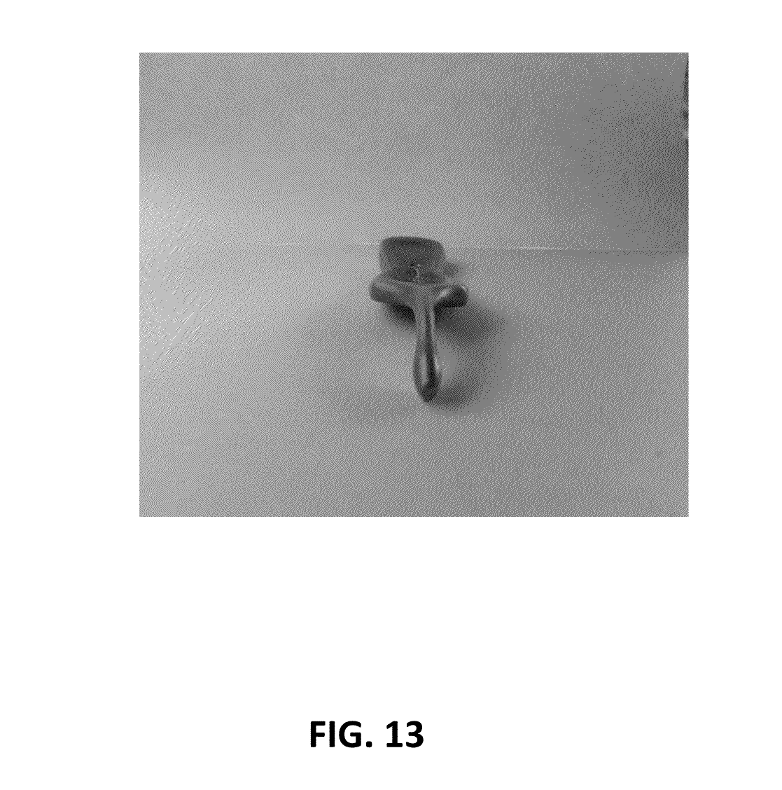

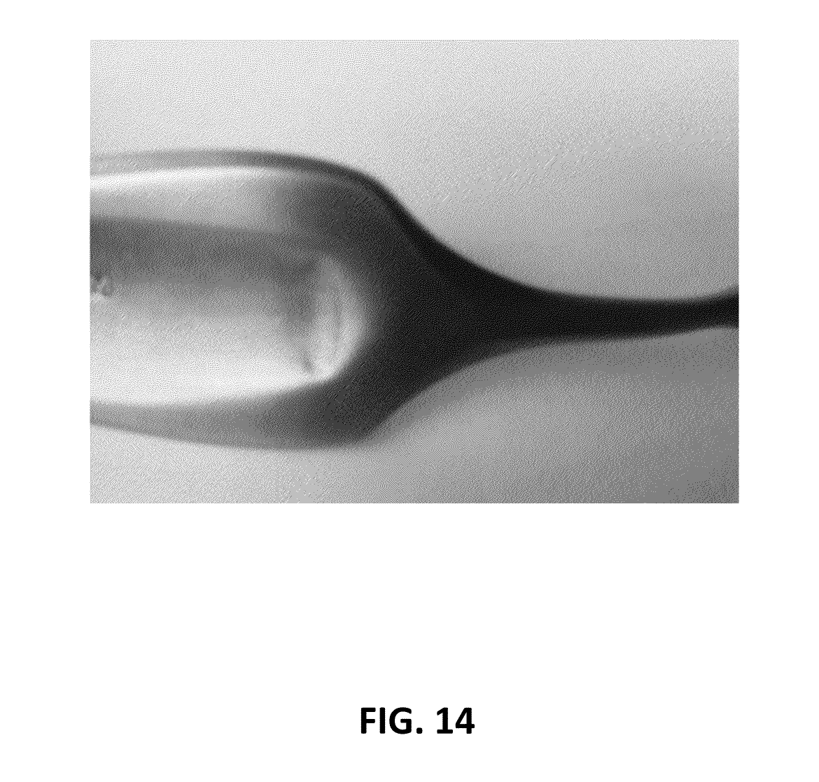

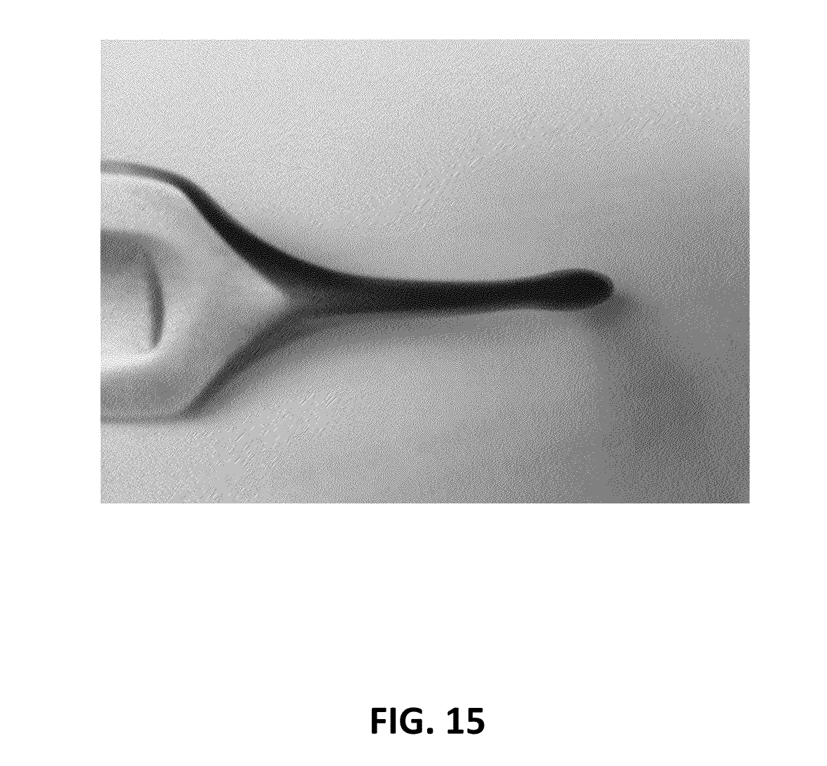

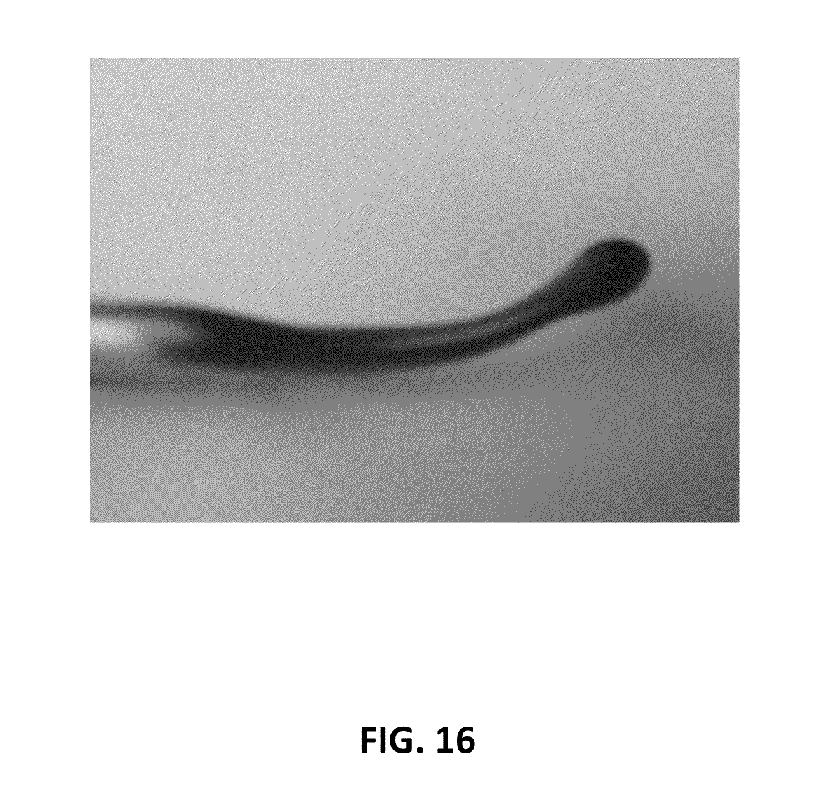

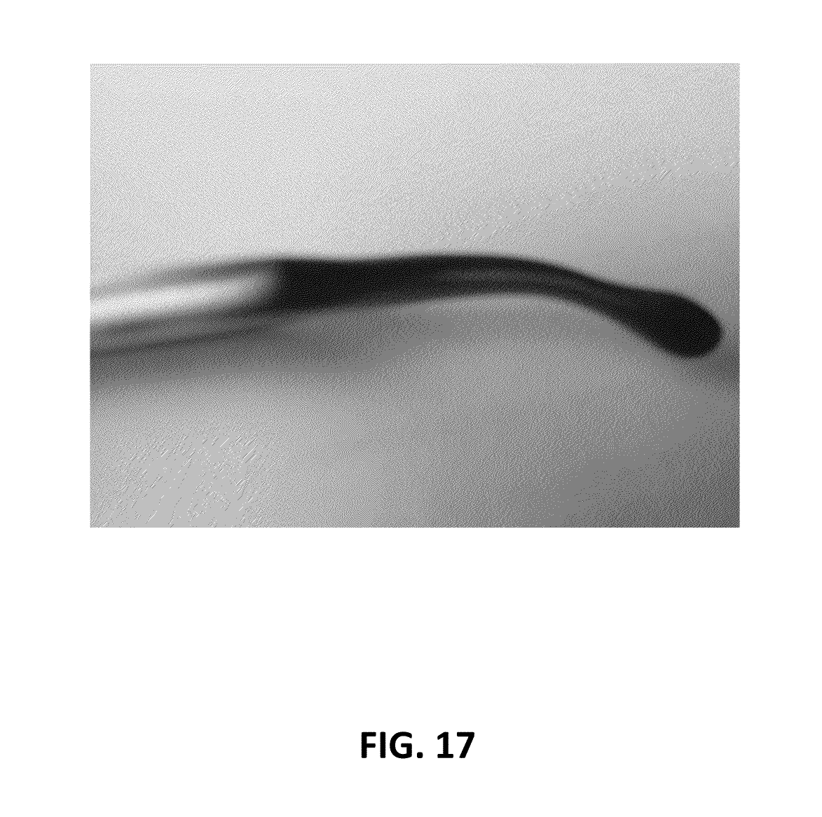

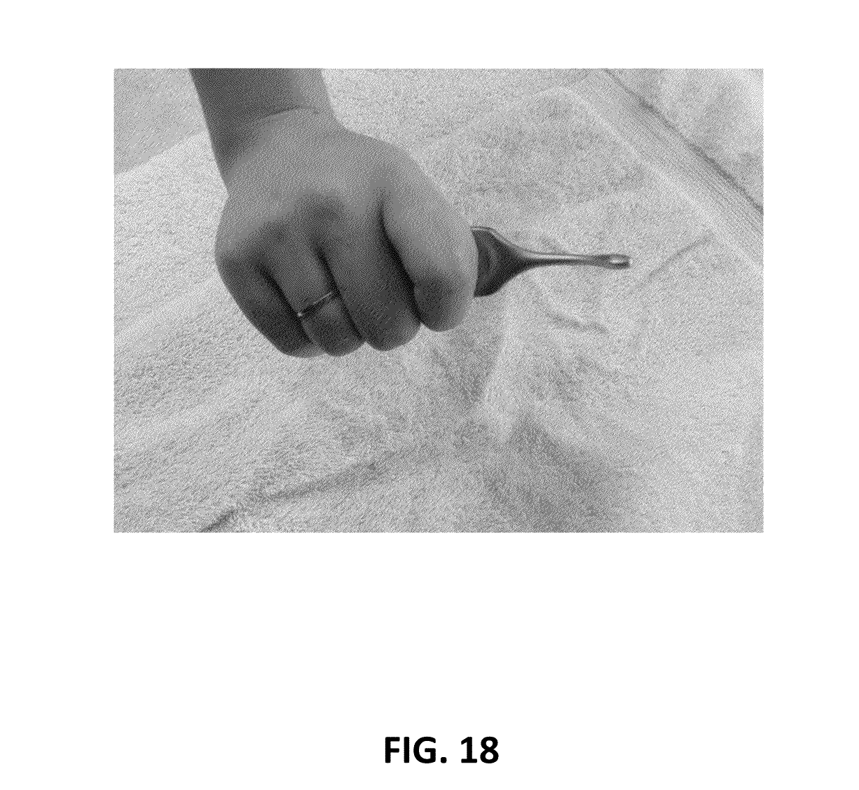

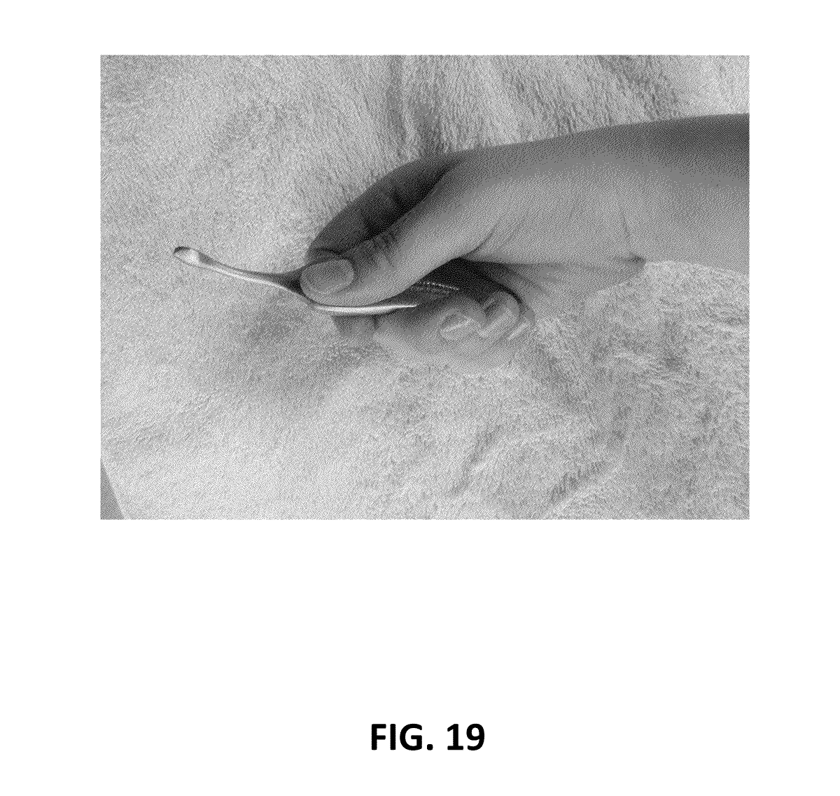

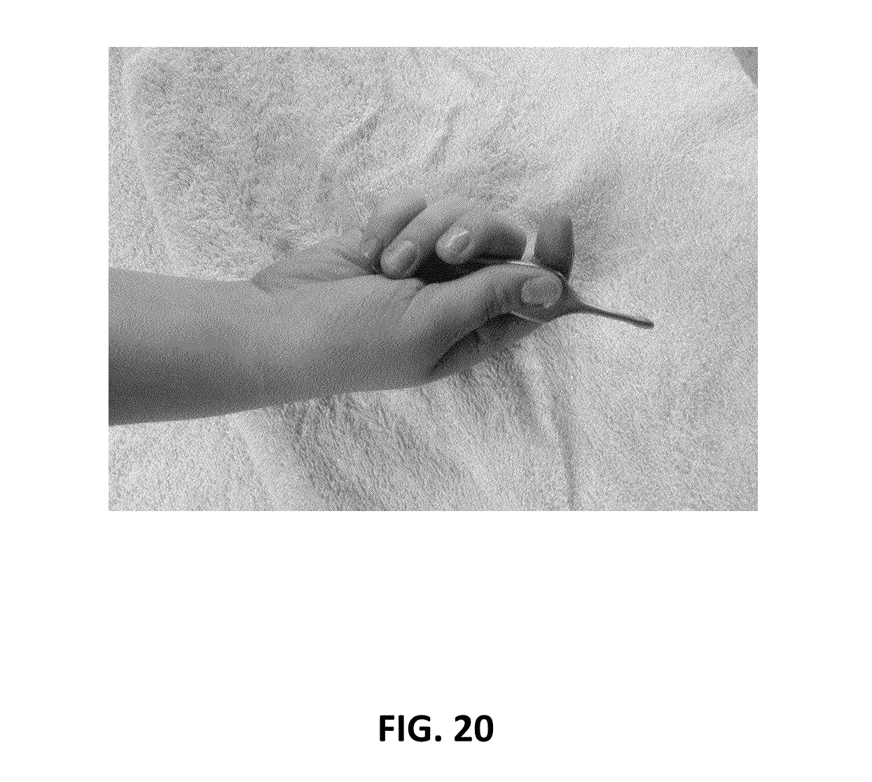

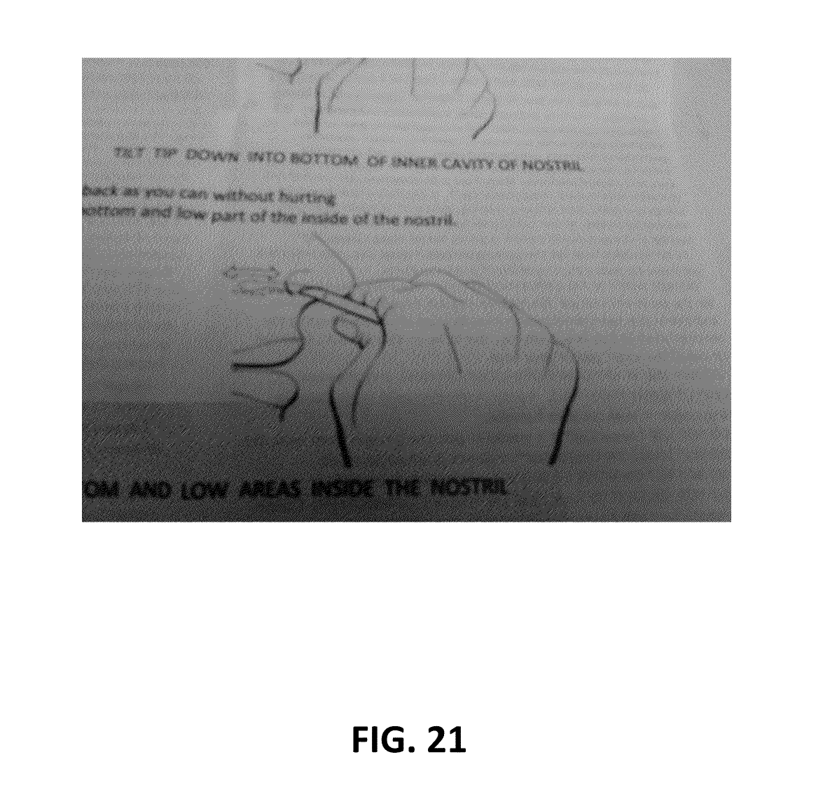

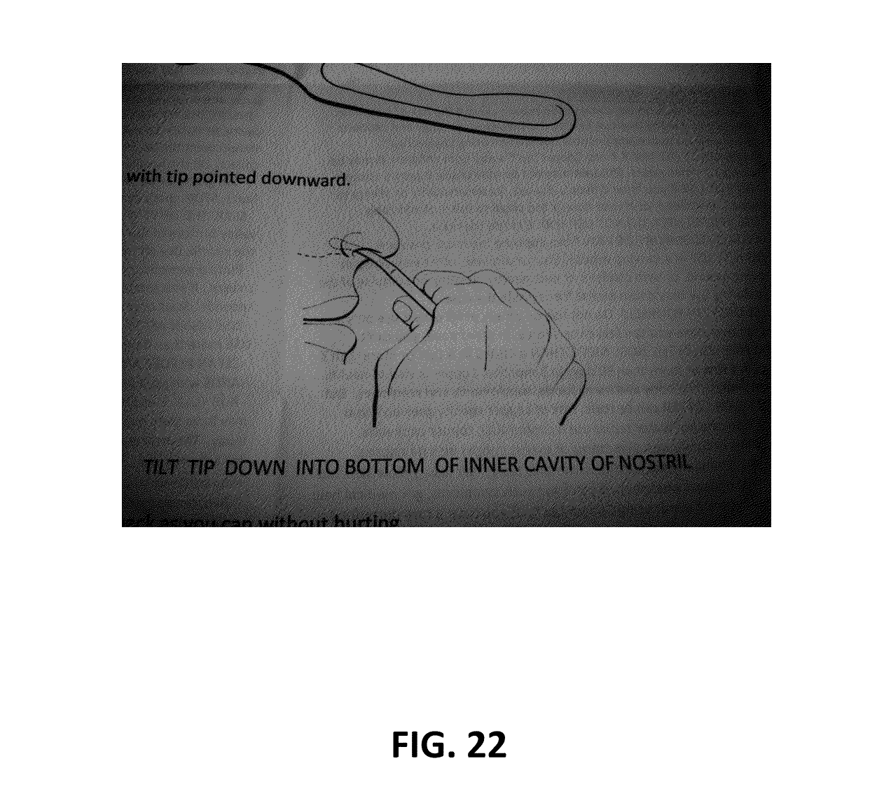

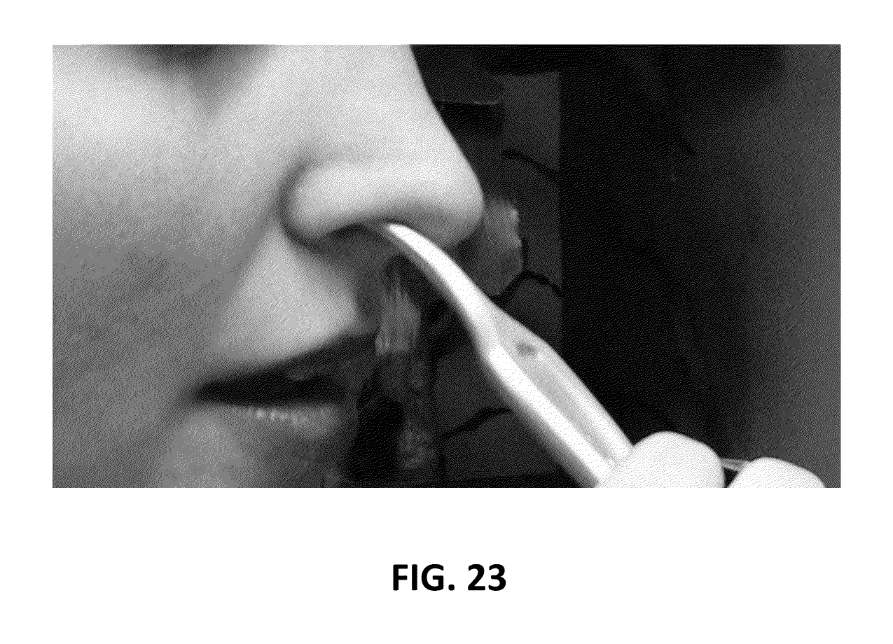

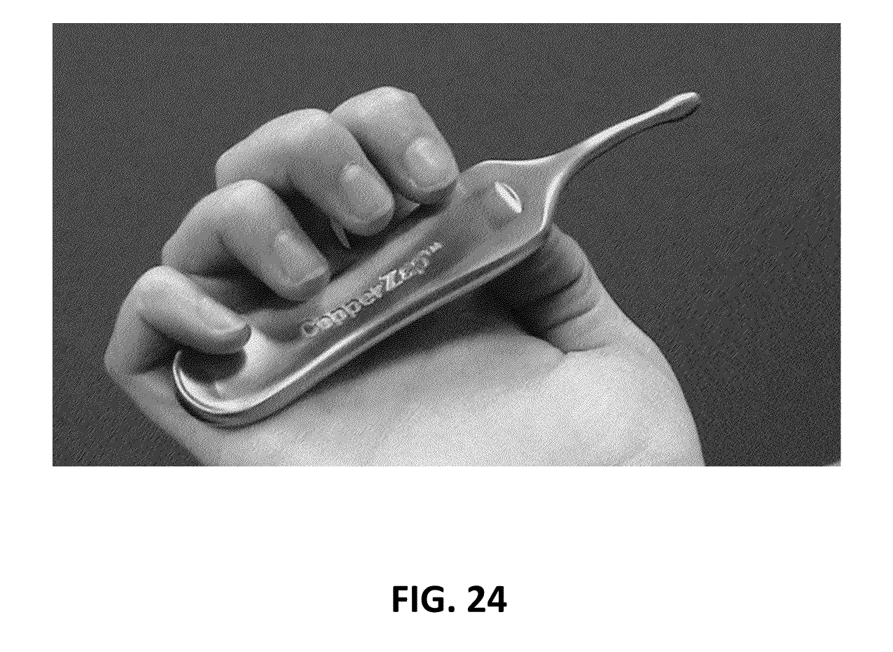

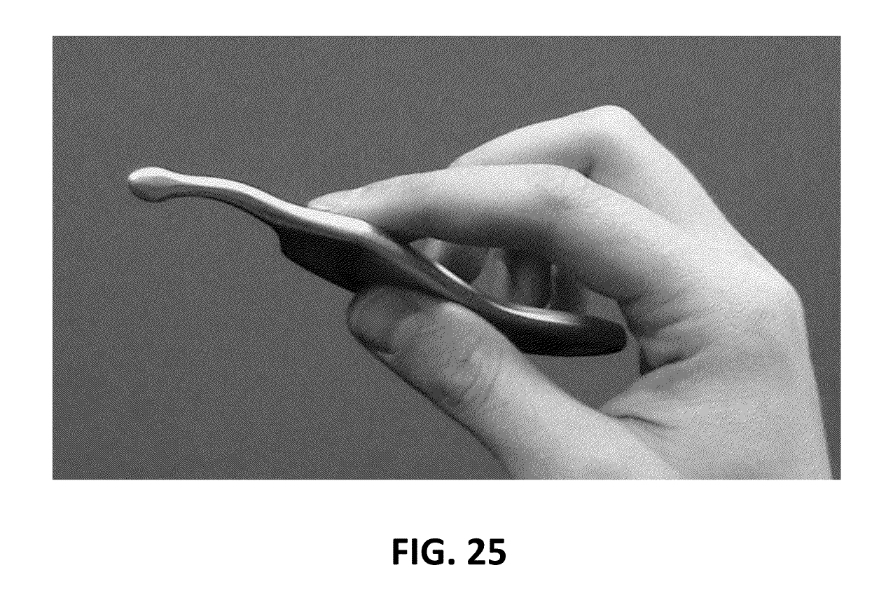

The substrate may be (or be a part of) a system or device adapted to be applied to the treatment area (e.g., mucosal area, vaginal area, anal area, etc.). Non-limiting examples of antimicrobial systems (e.g., devices) are described herein. For example, an antimicrobial system may comprise the substrate (with antimicrobial metal) attached to a handle. An example of an antimicrobial system adapted to be applied to a mucosal area is shown in FIG. 1 through FIG. 25. The system (100) features a handle (110) and a stem (120), wherein the stem (120) is effectively the substrate. The user can hold the handle (110) and insert the stem (120) (substrate) into the nasal cavity to directly contact the substrate with the appropriate tissues, linings, membranes, mucous, secretions, etc. The systems for mucosal area application are not limited to the configurations shown in FIG. 1 though FIG. 25.

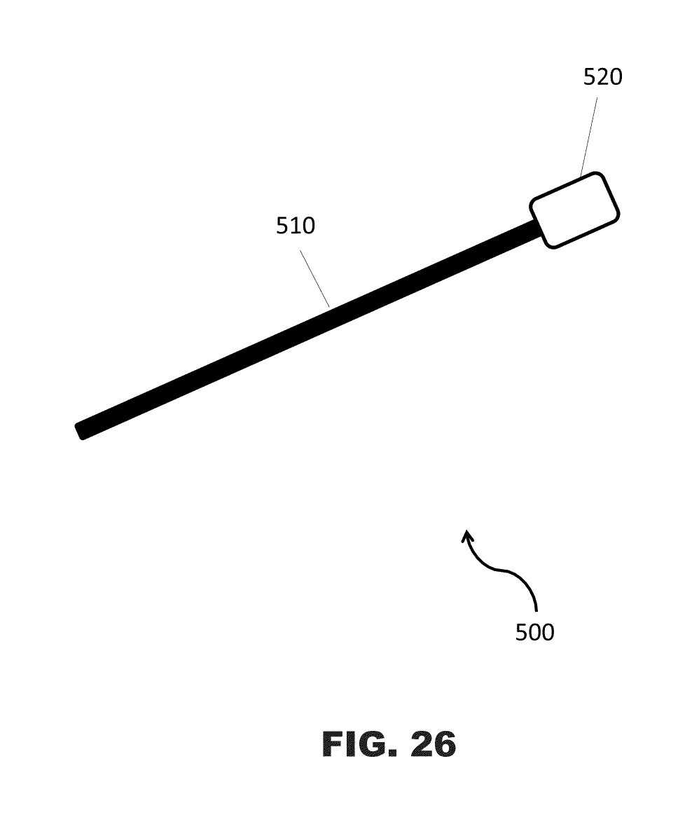

A basic (and non-limiting) example of an antimicrobial system adapted to be applied to a vaginal area is shown in FIG. 26. The system (500) features a handle (510) and a stem (520), wherein the stem (520) (e.g., a swab) is effectively the substrate. The user can hold the handle (510) and insert the stem (520) (substrate) into the vaginal area to directly contact the substrate with the appropriate tissues, linings, membranes, secretions, etc. The system (500) in FIG. 26 may alternatively be applied to an anal area. For example, the user can hold the handle (510) and directly contact the substrate with the appropriate tissues, linings, membranes, etc. In some embodiments, the system (500) may be inserted into the rectum to directly contact the substrate with the appropriate tissues, linings, membranes, etc.

The substrate is used to directly contact the pathogens residing in the treatment area. Directly contacting the pathogens residing in the mucosal area may comprise sliding, swabbing, or touching the substrate around the treatment area (e.g., the mucous, mucous membranes, vaginal area, cervical tissues, anal area, etc.). The substrate can be rubbed in/on the treatment area; however, simple direct contact may be sufficient, and the rubbing action may not necessarily be required (though possibly preferred by the user). The direct contact with the substrate (e.g., the antimicrobial metal of the substrate) is intended to kill or inhibit the pathogens in the treatment area.

Without wishing to limit the present invention to any theory or mechanism, it is believed that the step of directly contacting the treatment area (e.g., mucosal area, vaginal area, anal area, etc.) with the substrate comprising antimicrobial metal causes the deposit of a trace amount of antimicrobial metal ions onto the area of contact. The trace amount of antimicrobial metal ions may diffuse to other deeper tissues or secretions. This trace amount of antimicrobial metal ions may extend the killing/inhibiting process after the substrate is removed from the treatment area and enhance the effectiveness of the substrate by reaching pathogens not present on the surfaces that directly contacted the substrate. Without wishing to limit the present invention to any theory or mechanism, is may be possible that rubbing the substrate on/in the treatment area (as compared to simple direct contact) may increase the trace amount of antimicrobial metal ions deposited in the treatment area

### Antimicrobial Systems

Referring now to FIG. 1-25, the present invention also features antimicrobial systems (100) for application of an antimicrobial material on skin or mucous membranes or other treatment area. In some embodiments, the system (100) comprises a v-shaped base handle (110) having a stem end (111), a bottom end (112) opposite the stem end (111) having a radial terminating end, a base mid-section (200) between the stem end (111) and the bottom end (112), a front surface (114), a back surface (115) opposite the front surface (114), a base first side (206), a base second side (208) opposite the base first side (206), a base anterior handle component (202) from the stem end (111) to the base mid-section (200), and a base posterior handle component (204) from the bottom end (112) to the base mid-section (200).

In some embodiments, the base anterior handle component (202) is located at a base handle angle (210) to the base posterior handle component (204). In some embodiments, the base handle angle (210) is 5 degrees. In some embodiments, the base handle angle (210) is 10 degrees. In some embodiments, the base handle angle (210) is 15 degrees. In some embodiments, the base handle angle (210) is 20 degrees. In some embodiments, the base handle angle (210) is 25 degrees. In some embodiments, the base handle angle (210) is 30 degrees.

In some embodiments, the base handle (110) comprises a base anterior taper (212) from the stem end (111) to the base mid-section (200) on both the base first side (206) and the base second side (208). In some embodiments, the base handle comprises a base posterior taper (214) from the bottom end (112) to the base mid-section (200) on both the base first side (206) and the base second side (208). In some embodiments, a base mid-section width (216) from the base first side (206) to the base second side (208) is smaller than a base stem end width (218) from the base first side (206) to the base second side (208). In some embodiments, the base mid-section width (216) from the base first side (206) to the base second side (208) is smaller than a base bottom end width (220) from the base first side (206) to the base second side (208).

In some embodiments, the base mid-section width (216) is ¾ inch. In some embodiments, the base mid-section width (216) is ⅝ inch or less. In some embodiments, the base mid-section width (216) is ⅞ inch or greater. In some embodiments, the base stem end width (218) is ⅞ inch. In some embodiments, the base stem end width (218) is ¾ inch or less. In some embodiments, the base stem end width (218) is 1 inch or greater. In some embodiments, the base bottom end width (220) is ⅞ inch. In some embodiments, the base bottom end width (220) is ¾ inch or less. In some embodiments, the base bottom end width (220) is 1 inch or greater.

In some embodiments, the system (100) comprises a thumb indention (230) centrally located in the back surface (115) of the base anterior handle component (202) close to the stem end (111). In some embodiments, a thumb indention depth (232) tapers from a greatest depth close to the stem end (111) to flush with the back surface as the thumb indention (230) approaches the base mid-section (200). In some embodiments, a thumb indention depth (232) is 1/16 inch or less. In some embodiments, a thumb indention depth (232) is ⅛ inch or greater.

In some embodiments, the system (100) comprises a fingertip indention (240) centrally located in the back surface (115) of the base posterior handle component (204) close to the bottom end (112). In some embodiments, a fingertip indention depth (242) tapers from a greatest depth close to the bottom end (112) to flush with the back surface as the fingertip indention (240) approaches the base mid-section (200). In some embodiments, a fingertip indention depth (242) is 1/16 inch or less. In some embodiments, a fingertip indention depth (242) is ⅛ inch or greater.

In some embodiments, the system (100) comprises a concave indentation (118) located in the front surface (114) of the base handle (110) extending from at or near the stem end (111) to at or near the bottom end (112) of the base handle (110). In some embodiments, the concave indention (118) comprises a ridge (250) located there around bordering the concave indention (118). In some embodiments, a concave indention depth (252) tapers from a first concave indention depth (253) close to the stem end (111) to a second concave indention depth (254) greater than the first concave indention depth (253) at the base mid-section (200). In some embodiments, the concave indention depth (252) tapers from a third concave indention depth (255) close to the bottom end (112) to the second concave indention depth (254) greater than the third concave indention depth (255) at the base mid-section (200).

In some embodiments any one of a concave indention depth (252), a first concave indention depth (253), a second concave indention depth (254), and a third concave indention depth (255) are 1/16 inch or less. In some embodiments any one of a concave indention depth (252), a first concave indention depth (253), a second concave indention depth (254), and a third concave indention depth (255) are ⅛ inch. In some embodiments any one of a concave indention depth (252), a first concave indention depth (253), a second concave indention depth (254), and a third concave indention depth (255) are 3/16 inch or greater.

In some embodiments, the system (100) comprises an arcuate stem (120) extending upwardly from the stem end (111) of the base handle (110), the stem (120) has a distal end (121) and a close end (122) joined or coupled to the base handle (110). In some embodiments, the distal end (121) is rounded and is adapted for insertion into a nasal cavity. In some embodiments, the distal end (121) is bulbous.

In some embodiments, the distal end comprises a distal end width (260) geater than a stem width (270). In some embodiments, the distal end width (260) is 3/16 inch. In some embodiments, the distal end width (260) is ⅛ inch or less. In some embodiments, the distal end width (260) is ¼ inch or more. In some embodiments, the stem width (270) is ⅛ inch. In some embodiments, the stem width (270) is 3/16 inch or more. In some embodiments, the stem width (270) is 1/16 inch or less.

In some embodiments, the distal end comprises a distal end thickness (262) greater than a stem thickness (272). In some embodiments, the distal end thickness (262) is ⅜ inch. In some embodiments, the distal end thickness (262) is 5/16 inch or less. In some embodiments, the distal end thickness (262) is 7/16 inch or more. In some embodiments, the stem thickness (272) is ⅛ inch. In some embodiments, the stem thickness (272) is 1/16 inch or less. In some embodiments, the stem thickness (272) is 3/16 inch or more.

In some embodiments, a cross-section from a distal end top side (266) to a distal end bottom side (167) comprises a shape of an ellipse when viewed from a distal end first side (264) or a distal end second side (265). In some embodiments, a cross-section from the distal end first side (264) to the distal end second side (265) comprises a shape of a teardrop when viewed from a terminating distal end.

In some embodiments, at least a portion of an outer surface of the distal end (121) of the stem (120) and the base handle (110) comprises an antimicrobial material. In some embodiments, the stem (120) and the base handle (110) comprise unitary construction. In some embodiments, the antimicrobial material is solid copper or a copper alloy.

In some embodiments, the present invention features an antimicrobial sanitizer system (e.g., an antimicrobial applicator system (100)) for application of an antimicrobial material on a user's skin and/or mucous membranes. The system (100) comprises a base handle (110) and a stem (120) extending from the base handle (110). The stem (120) is optionally retractable into and extendable from the base handle (110). The base handle (110) may be used to apply antimicrobial material to a user's hands and fingers, face, or other body part. The stem (120) may be used to apply antimicrobial material to a treatment area, e.g., mucous membranes (e.g., where microorganisms lodge), such as the nasal cavity. In some embodiments, the antimicrobial material comprises copper. The antimicrobial material is not limited to copper.

The base handle (110) has a stem end (111) (e.g., a “shoulder”), a bottom end (112) opposite the stem end (111), a front surface (114), and a back surface (115). The directional terms “bottom,” “front,” and “back” (e.g., referring to the surfaces and ends of the base handle) are for reference only and are used for descriptive purposes only. The stem (120) extends from the stem end (111) of the base handle (110).

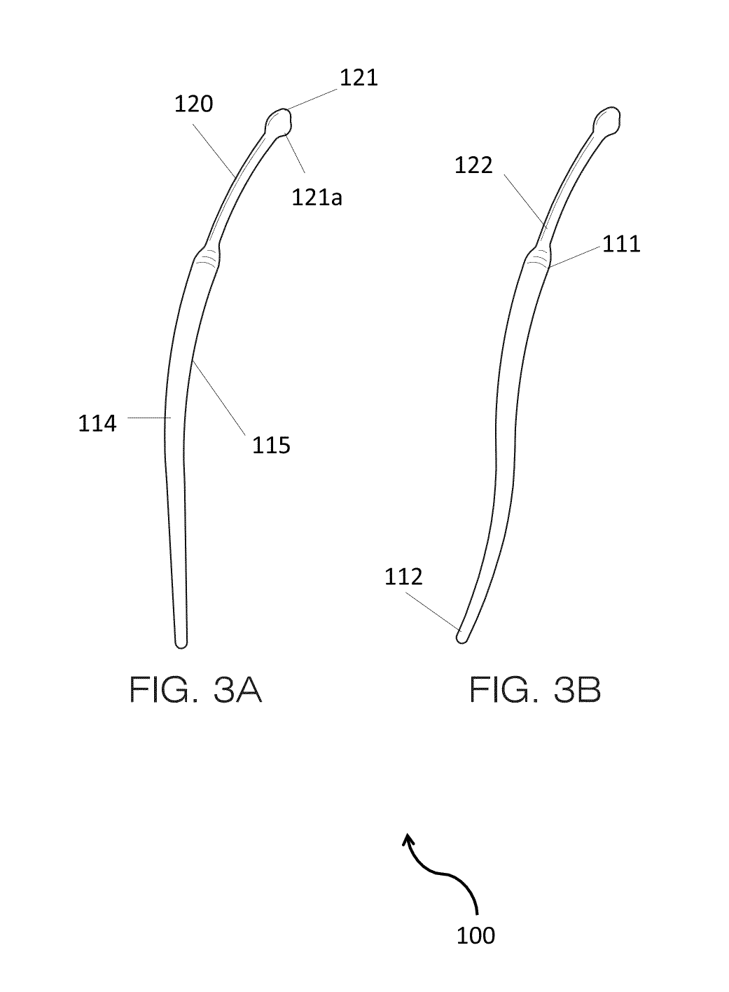

The base handle (110) may be constructed in a variety of shapes not limited to the shapes shown in the figures. For example, in some embodiments, the base handle (110) has rounded edges, for example for providing a comfortable rounded and smooth surface that can be rubbed on a user's face, hands (e.g., palms, fingers, etc.) or other body part. In some embodiments, the base handle (110) has an elongated shape, slightly rectangular with rounded edges. In some embodiments, the base handle (110) is arc-shaped (when viewed from the side, see FIG. 3A, FIG. 3B). In some embodiments, the base handle (110) is arc-shaped with two arcs in opposite directions (e.g., joined by an inflection point) as shown in FIG. 3B. In some embodiments, the stem end (111) of the base handle (110) has a width wider than that of the stem (120). In some embodiments, all or a portion of the base handle (110) has a width wider than that of the stem (120).

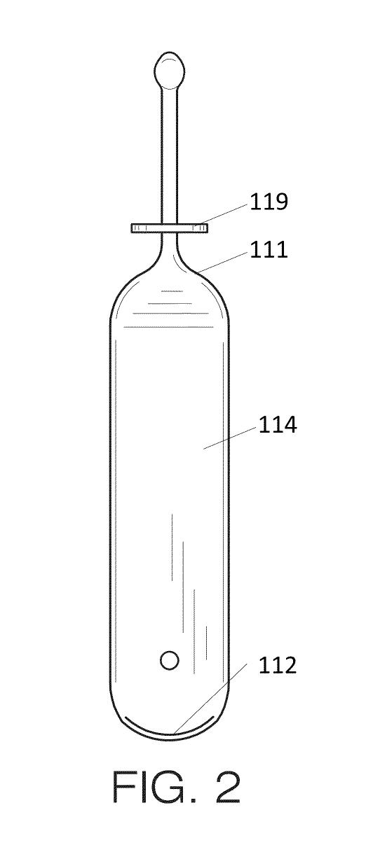

In some embodiments, the stem end (111) (e.g., shoulder) of the base handle (110) helps to restrict how far into the nasal cavity the stem (120) extends. This limitation can help prevent damage to the user's nasal cavity by extending the stem (120) too far into the nasal cavity. As shown in FIG. 2, in some embodiments, a stopper (119) is disposed on the stem (120), e.g., near the stem end (111) of the base handle (110) functioning to restrict how far into the nasal cavity the stem (120) extends. For example, in lieu of using the stem end (111) of the base handle (110) to restrict extension of the stem (120) into the nasal cavity, the stopper (119) may serve that purpose. In some embodiments, the stopper (119) is removable from the stem (120) (FIG. 1 shows the stopper (119) removed from the stem (120)).

The system (100) further comprises a stem (120) extending upwardly from the stem end (111) of the base handle (110). The stem (120) has a distal end (121) and a proximal end (122), the proximal end (122) being the end that is joined to or attached to (or coupled to) the base handle (110). The distal end (121) is rounded and is adapted for insertion into the nasal cavity, e.g., the distal end (121) is a bulb or knob. In some embodiments, the distal end (121) of the stem (120) is oval or spherical, however the distal end (121) of the stem (120) is not limited to an oval or spherical configuration. For example, in some embodiments, the distal end (121) of the stem (120) is spherical with a ridge or protrusion (121a) for reaching into a narrower or deeper portion of the nasal cavity. In some embodiments, the protrusion (121a) is disposed on the bottom surface of the distal end (121) of the stem (120), e.g., the end aligned with the bottom surface (115) of the base handle (110) (however, the position and orientation of the protrusion (121a) is not limited to the aforementioned configuration or the configurations shown in the figures). In some embodiments, the distal end (121) of the stem (120) has a width larger than that of the proximal end (122) of the stem (120). In some embodiments, the distal end (121) of the stem (120) has a width larger than that of the middle portion of the stem (120), e.g., the portion in between the proximal end (122) and the distal end (121).

At least a portion of the outer surface of the distal end (121) of the stem (120) comprises an antimicrobial material. For example, in some embodiments, the stem (120) is constructed entirely from the antimicrobial material (e.g., copper). In some embodiments, the stem (120) is constructed from a material that is coated with the antimicrobial material (e.g., copper). In some embodiments, the entire outer surface of the stem (120) features exposed antimicrobial material (e.g., copper). In some embodiments, only a portion of the outer surface of the stem (120) features exposed antimicrobial material (e.g., copper).

In some embodiments, at least a portion of an outer surface of the base handle (110) comprises an antimicrobial material. For example, in some embodiments, the base handle (110) is constructed entirely from the antimicrobial material (e.g., copper). In some embodiments, the base handle (110) is constructed from a material that is coated with the antimicrobial material (e.g., copper). In some embodiments, the entire outer surface of the base handle (110) features exposed antimicrobial material (e.g., copper). In some embodiments, only a portion of the outer surface of the base handle (110) features exposed antimicrobial material (e.g., copper).

The base handle (110) and stem (120) may be constructed in a variety of configurations. For example, in some embodiments, the base handle (110) and/or the stem (120) are constructed as a solid piece, for example a solid piece of copper, a solid piece of material (e.g., plastic) coated with copper. In some embodiments, the base handle (110) and/or the stem (120) are constructed as a coil, e.g., a copper coil.

In some embodiments, the base handle (110) is curved or arc-shaped (see FIG. 3A, FIG. 3B). In some embodiments, the stem (120) is curved or arc-shaped (see FIG. 3A, FIG. 3B). The curvature of the stem (120) may help the distal end (121) of the stem (120) to reach a particular portion of the nasal cavity, for example the inferior meatus.

In some embodiments, the base handle (110) is arc shaped and has an arc length of between about 3 to 8 inches, for example about 3 inches, about 3.5 inches, etc. In some embodiments, the base handle (110) is arc shaped and has an arc length of between about 4 to 10 inches. In some embodiments, the base handle (110) is arc shaped and has an arc length of between about 4 to 6 inches. The arc length of the base handle (110) is not limited to the aforementioned examples. For example, in some embodiments such as the embodiments with the retractable stem (120) described below, the base handle (110) has an arc length of about 3.5 inches.

In some embodiments, the base handle (110) is arc shaped and has radius of curvature of between about 5 to 14 inches, e.g., 7 inches, 5.5 inches, etc. In some embodiments, the base handle (110) is arc shaped and has radius of curvature of between about 8 to 20 inches. In some embodiments, the base handle (110) is arc shaped and has radius of curvature of between about 6 to 30 inches. In some embodiments, the base handle (110) is arc shaped and has radius of curvature of between about 10 to 30 inches. The radius of curvature of the base handle (110) is not limited to the aforementioned examples.

In some embodiments, the stem (120) is arc shaped and has an arc length of between about 0.5 to 5 inches, for example about 2 inches, about 3 inches, etc. In some embodiments, the stem (120) is arc shaped and has an arc length of between about 1 to 3 inches. In some embodiments, the stem (120) is arc shaped and has an arc length of between about 1.5 to 3 inches. The arc length of the stem (120) is not limited to the aforementioned examples. For example, in some embodiments such as the embodiments with the retractable stem (120) described below, the stem (120) has an arc length of about 3 inches overall but only about 2 inches of the stem (120) extend beyond the base handle (110).

In some embodiments, the stem (120) is arc shaped and has radius of curvature of between about 4 to 12 inches, e.g., 5 inches. In some embodiments, the stem (120) is arc shaped and has radius of curvature of between about 8 to 20 inches. In some embodiments, the stem (120) is arc shaped and has radius of curvature of between about 6 to 30 inches. In some embodiments, the stem (120) is arc shaped and has radius of curvature of between about 10 to 30 inches. The radius of curvature of the stem (120) is not limited to the aforementioned examples. In some embodiments, the curvature radius of the stem may not be constant, e.g. the radius may vary.

In some embodiments, the base handle (110) may not have a constant radius of curvature. For example, in some embodiments, one portion of the base handle (110) may have a first radius of curvature and a second portion of the base handle (110) may have a second radius of curvature. In some embodiments, the base handle (110) has an S-shape wherein the direction of the curvature changes (e.g., via an inflection point) (see FIG. 3B). In some embodiments, the stem (120) is part of the S-shape, e.g., the S-shape includes the stem (120).

In some embodiments, the stem (120) may not have a constant radius of curvature. For example, in some embodiments, one portion of the stem (120) may have a first radius of curvature and a second portion of the stem (120) may have a second radius of curvature.

In some embodiments, the stem (120) and the base handle (110) are both arc shaped and the stem (120) has a radius of curvature that is different from that of the base handle (110).

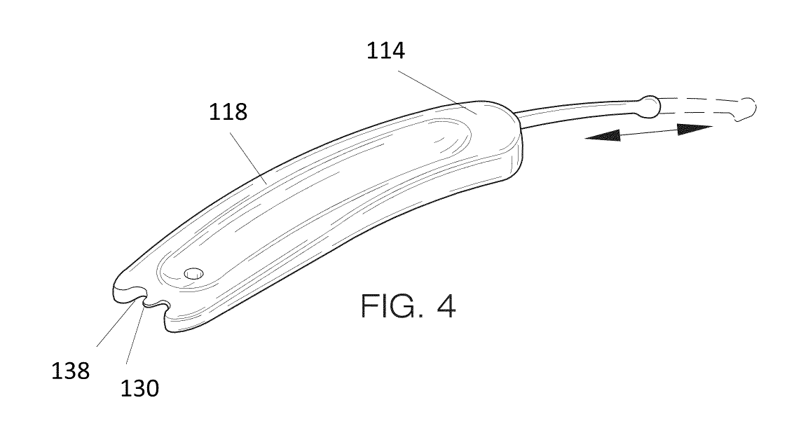

As shown in FIG. 4, in some embodiments, the front surface (114) of the base handle (110) is curved inwardly, e.g., the front surface (114) comprises a concave indentation (118). The concave indentation (118) may extend from at or near the stem end (111) to at or near the bottom end (112) of the base handle (110). The concave indentation (118) may function to provide additional surface area to the front surface (114) of the base handle (110), e.g., to provide more area on which a user can rub his/her fingers, palms, or other body part. A user may choose to rub his/her hands and/or fingers along the front surface (114), e.g., the concave indentation (118) and/or the back surface (115) of the base handle (110).

In some embodiments, the system (100) further comprises a concave indentation disposed in the back surface (115) of the base handle (110), e.g., concave indentations are disposed in both the front surface (114) and back surface (115) of the base handle (110). The concave indentation (118) in the back surface (115) may extend from at or near the stem end (111) to at or near the bottom end (112) of the base handle (110). The concave indentation (118) in the back surface (115) of the base handle (110) may function to provide additional surface area to the back surface (115) of the base handle (110), e.g., to provide more area on which a user can rub his/her fingers, palms, or other body part. In some embodiments, a user may use one indentation for his/her thumb and the opposite indentation for his/her fingers.

As shown in FIG. 4, in some embodiments, the system (100) further comprises a flange (130) extending from a surface of the base handle (110). For example, in some embodiments, the flange (130) extends downwardly from the bottom end (112) of the base handle (110). In some embodiments, the flange (130) extends outwardly from a side of the base handle (110). The flange (130) is not limited to placement on the bottom end (112) or a side of the base handle (110) and may be situated on any appropriate surface of the base handle (110). The flange (130) is adapted to be inserted under a user's fingernail or other appropriate location. In some embodiments, an inward curvature (138) is disposed at the location of the flange (130) and the flange (130) extends from the inward curvature. For example, in some embodiments, the inward curvature (138) is disposed in the bottom end (112) of the base handle (110) and the flange (130) extends downwardly from the inward curvature (138). In some embodiments, the flange (130) does not extend beyond the bottom end (112) of the base handle (110) or the adjacent surface of the base handle (110) depending on the location of the flange (130).

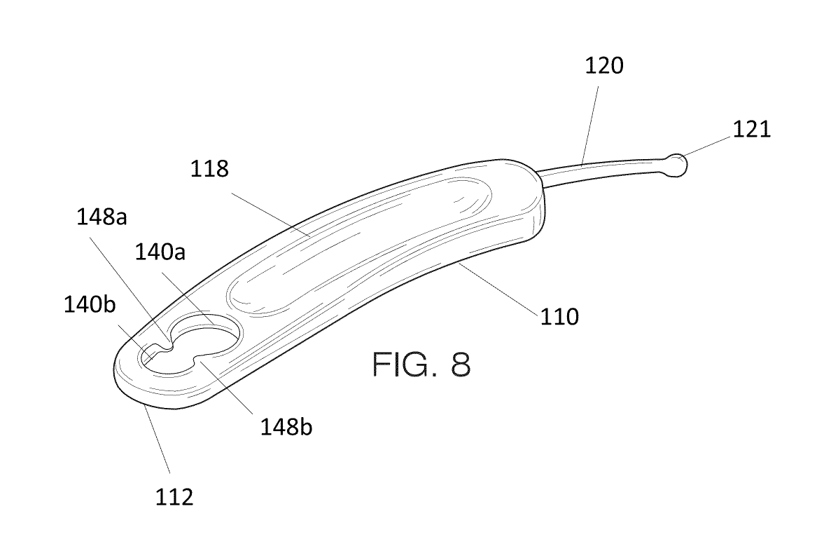

In some embodiments, the system (100) further comprises a hole (140) disposed in the base handle (110) adapted to accept a string or a lanyard. The hole (140) may be constructed in a variety of shapes and sizes. For example, in some embodiments, the hole (140) is circular in shape (as viewed from the front surface (114) as shown in FIG. 2. In some embodiments, the hole (140) is an alternative shape as shown in FIG. 8. For example, the hole (140) may have a generally hourglass shape (as shown in FIG. 8). For example, the hole (140) may comprise two cavities, e.g., two opposing cavities, e.g., a first cavity (140a) and a second cavity (140b). The cavities (140) may be separated by a first cavity protrusion (148a), which extends a distance into the hole (140). In some embodiments, the cavities (140) are also separated by a second cavity protrusion (148b), which extends a distance into the hole (140). In some embodiments, the cavity protrusions (148) are opposite each other (as shown in FIG. 8). In some embodiments, the cavity protrusions (148) are not opposite each other. The hole (140) may be constructed in any appropriate shape and is not limited to the examples shown in the figures.

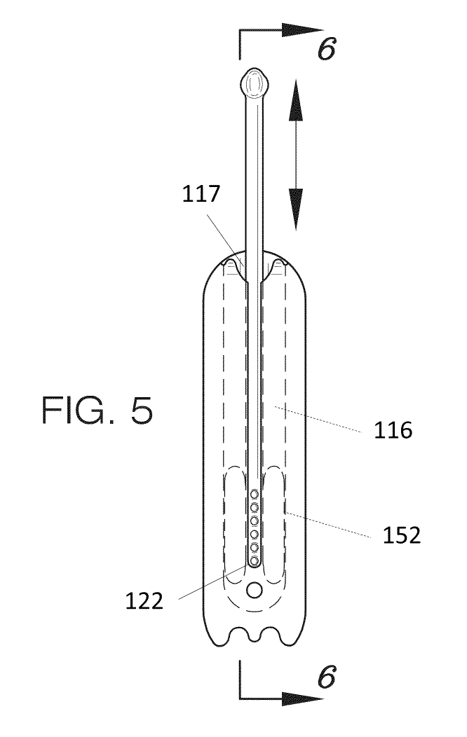

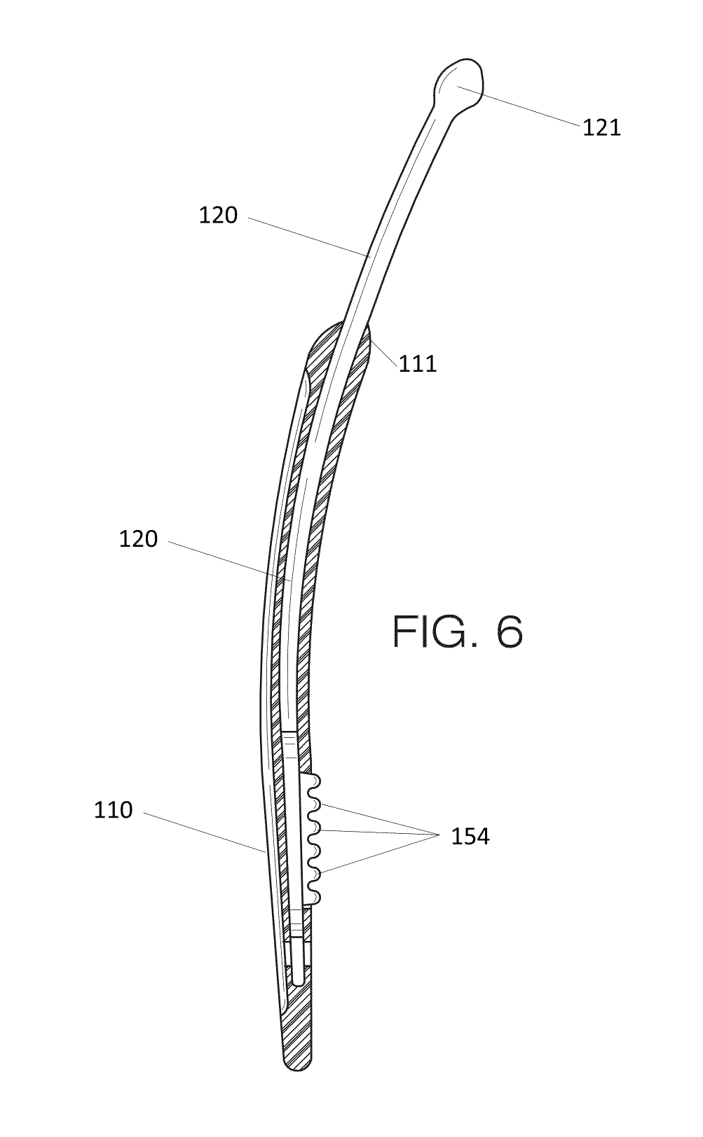

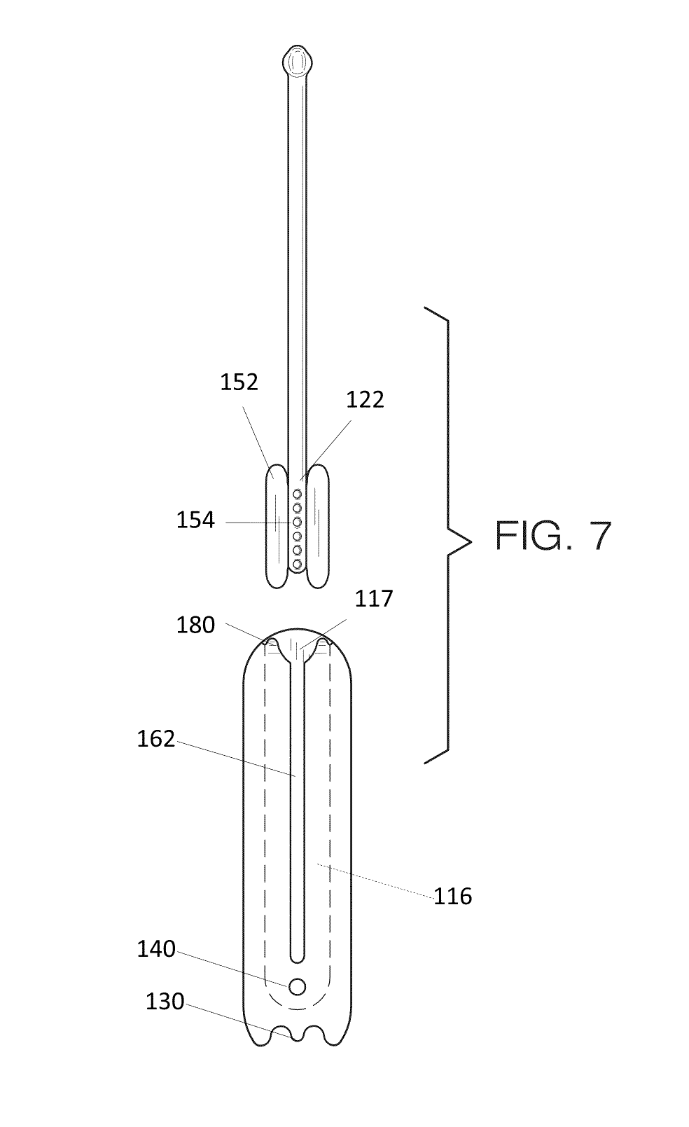

The antimicrobial applicator system may feature a retractable and extendable stem (120) coupled to the base handle (110). For example, as shown in FIG. 5, FIG. 6, and FIG. 7, in some embodiments, the base handle (110) has an inner cavity (116), which is accessible via an opening or aperture (117) disposed in the stem end (111) of the base handle (110). The inner cavity (116) is adapted to accept the stem (120), e.g., the proximal end (122) of the stem (120). For example, the proximal end (122) of the stem (120) may be slidably engaged in the aperture (117) and inner cavity (116) of the base handle (110). In some embodiments, the stem (120) is extendable from the stem end (111) of the base handle (110) and retractable into the inner cavity of the base handle (110).

As shown in FIG. 7, in some embodiments, a slit (162) is disposed in the front surface (114) of the base handle (110) and extends from the aperture (117) towards the bottom end (112) of the base handle (110). The slit (162) has a width smaller than that of the inner cavity (116) of the base handle (110). In some embodiments, the slit (162) has a width smaller than that of the stem (120). In some embodiments, one or more friction nubs (154) are disposed on the front surface of the stem (120) (e.g., at or near the proximal end (122)) wherein the friction nub(s) (154) protrude through the slit (162). The friction nubs (154) may provide a surface for helping a user grip the stem (120) to allow retraction and extension of the stem (120). For example, a user can slide the friction nub (154) upwardly and downwardly within the slit (162) to respectively extend the stem (120) from and retract the stem (120) into the base handle (110). FIG. 6 shows the friction nubs (154) protruding through the slit (162).

In some embodiments, a tongue (152) extends outwardly from one or both sides of the proximal end (122) of the stem (120). The tongue (152) engages the inner cavity (116) of the base handle (110). The tongue (152) helps retain the stem (120) in the inner cavity (116) of the base handle (110) in the presence of the slit (162). For example, because the slit (162) is narrower than the width of the stem (120) with the tongue (152), the stem (120) with the tongue (152) is retained in the inner cavity (116) of the base handle (110).

As shown in FIG. 5 and FIG. 7, in some embodiments, the base handle (110) comprises retention tabs (180) for helping prevent the stem (120) (e.g., with tongue) from sliding all of the way out of the inner cavity (116) and separating from (disengaging) the base handle (110). In some embodiments, retention tabs (180) are disposed on the front surface (114) of the base handle (110) surrounding the aperture (117). For example, a first retention tab (180) may be disposed at the intersection of the front surface (114) of the base handle (110) and the aperture (117) on one side of the slit (162). A second retention tab (180) may be disposed at the intersection of the front surface (114) of the base handle (110) and the aperture (117) on a second side of the slit (162). The retention tabs (180) may fold toward the back surface (115) of the base handle, e.g., over the inner cavity, over the aperture (117), etc.

In some embodiments, the stem (120) is flexible to help the retraction into the base handle (110) and extension from the base handle (110).

In some embodiments, the stem (120) is free from the base handle (110). For example, the system (100) may comprise a stem (120) with a distal end (121) and a proximal end (122).

In some embodiments, the top (the distal end (121) of the stem (120)) is bulged. The bulge may help provide more surface area to the tip of the stem (120) so as to increase the area of the inferior meatus that can be contacted with the stem (120). The bulge may be biased and positioned on the concave side of the arched stem.

As described above, the stem (120) may be arched (e.g., having an arc length, a radius of curvature). The arch shape may help to allow the swabbing of the inferior meatus and avoiding the superior meatus.

The system (100) of the present invention is portable and provides a personal touch-surface. The system (100) also allows for active use. Without wishing to limit the present invention to any theory or mechanism, it is believed that the active use of copper or other antimicrobial material (and/or the system) is advantageous because it allows for direct contact with a large portion of the pathogen population present in the area of application and also because it increases the spreading and diffusion of antimicrobial ions through the media (e.g., nasal mucous, etc.), and also it is believed that the active use of copper (and/or the system) is advantageous because it allows for more/deeper contact (and longer contact) with the pathogens, e.g., via a diffusion process. It may allow for the spreading of copper ions through the media (e.g., nasal mucous, etc.).

In some embodiments, the methods of the present invention comprise obtaining an antimicrobial system such as the ones described herein (e.g., antimicrobial applicator system (100)). The methods may comprise rubbing one or more fingers and a thumb of a user on a base handle (110) to deposit a residue of antimicrobial material thereon. In some embodiments, the methods comprise gripping the base handle (110) via wrapping one or more fingers around the base handle (110) and placing the thumb of the user into a thumb indention (230). In some embodiments, an index finger of the user is placed against a front surface (114) of the base handle (110) wrapping around a base first side (206) and a base second side (208). In some embodiments, pressure is applied from the index finger and the thumb of the user to securely hold the base handle (110). In some embodiments, the methods comprise placing a distal end (121) of the stem (120) into an inner cavity of a nostril of the user. In some embodiments, the distal end (121) is moved around in contact with a bottom floor of the inner cavity of the nostril swabbing the inner cavity of the nostril to deposit a residue of antimicrobial material thereon. In some embodiments, the methods comprise placing a surface of the base handle (110) on an exterior surface of a nose and face of the user and swabbing to deposit a residue of antimicrobial material thereon.

In some embodiments, the system of the present invention is used as a pre-surgery application.

For any of the methods described herein, the stem may be applied to swab the outer cavity (vestibule) in the nostril. In some embodiments, the stem may be applied to swab the inner cavity of the nostril. In some embodiments, the stem may be applied to swab the inferior meatus region. In some embodiments, the stem may be applied to swab the superior meatus region. In some embodiments, the stem may be applied to swab only one of the regions discussed above or more than one of the regions discussed above.

In some embodiments, the present invention comprises a stem that is free of a base. In some embodiments, the stem is connected to a small base. The small base may be a round disk-like structure.

Generally, the system may be applied to the treatment area quickly (e.g., the system does not need to be held in place for any long period of time). However, in some embodiments, the system (e.g., stem) may be applied to swab the treatment area (e.g., nostril regions mentioned above) for about 5-10 seconds, about 10 to 20 seconds, about 30 to 40 seconds, about 40 to 50 seconds, about 50 to 60 seconds or more, or as desired.

In some embodiments, the swabbing of the stem in the nostril as disclosed herein may be effective to eliminate microbes responsible for the common cold, the flu, or other disease causing pathogens including Methicillin-resistant Staphylococcus aureus (MRSA), Influenza A virus, etc.

Without wishing to limit the invention to any mechanism of action, it is believed that depositing copper residue is a secondary effect of the action to killing the pathogen. The primary effect may be the direct contact of the antimicrobial material (e.g., copper) with the microbes, which is believe to be what accomplishes much if not most of the killing of pathogens.

As used herein, the term “about” refers to plus or minus 10% of the referenced number. For example, an embodiment wherein the stem has an arc length of about 2 inches includes a stem with an arc length of between 1.8 and 2.2 inches.

### Example 1

### Use of Sanitizer System in Hospital Setting

A Nurse or Doctor carries the presently claimed Sanitizer System in her coat jacket pocket or on a lanyard around her neck. The System is made of a copper alloy recognized by the Environmental Protection Administration (EPA) as having antimicrobial properties in certain applications.

On the way to a ward or to a patient's room, the Nurse or Doctor rubs her fingers and thumb on the Sanitizer System, which is believed to help kill any pathogens that may be present, thus reducing her chance of transmitting a live pathogen to a patient. The rubbing also lays down a residue of copper, which is believed to help kill any pathogens she may pick up from a patient or from items she touches in the room. She may also swab the tip of the wand in the vestibule of her nostrils and rub the edges of the base portion of the system on her nose and face to further lay down a residue of copper alloy. This may further reduce the chance of her acquiring an infectious illness in case she touches her face with a fingertip that might at that moment be host to a live pathogen.

The curvature of the front and back of the base, or handle, of the System increases the surface area of contact between the antimicrobial material of the surface of the System and the skin of the user. The design is intended to maximize such contact when the System is held in the hand. In addition, the curvatures of the edges of the base, or handle, are designed to make it comfortable and easy to rub against the face and nose, including the skin over the cheekbones, the lips, and the underside of the nostrils, in a manner that can effectively reach all the facial surfaces, concave, convex, and otherwise. The flanges in some embodiments of the System are designed to allow rubbing of the antimicrobial material under the fingernails and thumbnails with maximum convenience. The entire shape of the System is designed to allow one-handed use, while walking, by a busy health care worker who may be carrying things in the other hand and who is likely to be in a hurry.

Most infectious illnesses are transmitted by touch. Since people naturally touch their face unconsciously many times a day, it is easy for a pathogen to travel from fingertip to face and thence to the nose and into the body. A residue of antimicrobial material on fingers, face and nose may inhibit this route of transmission. In addition, it may reduce the chance she may transmit the pathogen to others. In addition, application of the System in the nostril may inhibit the acquisition of airborne pathogens.

Immediately after contact with the patient, if the Nurse or Doctor does not have an opportunity to wash her hands right away, she rubs the Sanitizer System again to increase the potential kill rate of any pathogens she may have picked up. The residue is absorbed by skin and mucous membranes over a period of about half an hour, so it is only a temporary shield and should be renewed frequently by further rubbing.

Use of the System in a health care setting is an addition to, rather than a substitute for, EPA-recommended use of Antimicrobial Copper (trademark of the Copper Development Association) in “touch-surfaces” in hospitals, such as bedrails, doorknobs, call buttons, faucets, light switches, etc. Tests by the EPA and others show Antimicrobial Copper inhibits or kills, on contact in minutes, a wide variety of dangerous bacteria and viruses, including MRSA, VRE, C. Diff, E. coli, Influenza A, H1N1, Rhinovirus, Adenovirus, and others.

### Example 2

### Use of Sanitizer System while Traveling on Public Commuter Systems

A passenger carries the Sanitizer System on his person. Just prior to entering a plane, train, or bus, he uses it in a similar manner to the Nurse or Doctor in Example 1, at least on the fingers and thumbs. He uses it again just before and just after touching various surfaces known to harbor potential pathogens, such as airplane tray tables and lavatory fixtures. Assiduous use may reduce the chance not only of acquiring pathogens but also of spreading them.

### Example 3

### Use of Sanitizer System while Shopping

In the process of shopping, a person comes in contact with a wide array of microbes on everything from items for sale to credit cards. Paper money is especially known to carry many bacteria and viruses. Even if a shopper doesn't use paper money, she touches objects that have been handled by store clerks who, as part of their job, must handle paper money from many other customers, some of whom may be carrying infectious illnesses.

The shopper may use the system on fingers and thumbs in a manner similar to the Nurse or Doctor in Example 1 before and after shopping and before and after each instance of handling money. Shoppers may be disinclined to use the System of their faces or in their nostrils in public, but it may be less important to do so in a store than in a health care setting, especially if the shopper makes an effort to avoid touching her face until after she returns home and washes her hands thoroughly. Use of the System is never a substitute for regular and thorough hand-washing.

### Example 4

### Use of Sanitizer System in Day Care and School Settings

A day care worker or a schoolteacher may use the System in a manner similar to the Nurse or Doctor in Example 1 at frequent intervals through the workday. In addition to potentially protecting themselves and the children in their care, the worker or teacher may also reduce the risk of bringing home infectious pathogens and spreading illness to their own families

### Example 5

### Use of Sanitizer System in Elder Care Facilities

It is known that outbreaks of flu or other infectious illness in elder care facilities may start with one visitor or staff member and spread to residents, then to other staff members, and to other visitors and their families. Staff members and visitors may use the System in a manner similar to the Nurse or Doctor in Example 1 before and after contact with residents, to potentially help protect themselves and everyone else involved.

### Example 6

### Use of Sanitizer System to Fight Off the Common Cold or Flu

A cold or flu often starts with a tickle in the nose, which signals the accumulation of Rhinovirus or Adenovirus in the inner cavity of the nostril. Sometimes cold or flu may start with a scratchy throat, which signals the same thing and results from virus-containing mucous dripping from the nostril backwards into the throat. Upon any early sign of cold or flu, a user may gently rub the tip of the wand of the Sanitizer System in the inner cavity of the nostril, especially in the bottom of the cavity, known as the inferior meatus, where the cold or flu virus may first accumulate and replicate. The copper begins killing the cold or flu virus immediately. The copper residue left after 30 seconds or a minute of rubbing is believed to continue killing the virus after the wand is removed. The effect is believed to be similar to that of zinc-containing gel, once sold under the trade name Zicam. Zinc has antimicrobial properties akin to those of copper. Both copper and zinc kill cold and flu viruses on contact in minutes. If used early enough, they are believed to reduce the virus population in the nostril and inhibit its replication. Suppression of the virus numbers can allow the immune system to gain the upper hand quickly and fully defeat the cold or flu in the first day or two, before any major symptoms develop.

Despite its effectiveness, Zicam was removed from the market by the FDA after it was determined that Zicam could damage the sense of smell of the user, sometimes permanently. It is believed that the damage resulted from inhalation of quantities of the zinc gel, which drew significant quantities of zinc deep into the nostril and brought it into contact with the olfactory nerves, which control the sense of smell. The Sanitizer System limits the length of the wand so that it cannot reach the olfactory nerves and does not deposit significant quantities of material, thus avoiding the threat to the sense of smell. The System is not recommended for use by persons under the age of 12, however, due in part to the risk of reaching the tip of the wand all the way to the olfactory nerves in a child-sized nose, just in case the slight copper residue might damage the nerves.

In some embodiments, the System may use zinc instead of copper. Copper, however, may be more effective than zinc due in part to greater electrical conductivity, which is thought to be a factor in its anti-pathogen effect. Copper is also believed to have a higher threshold of toxicity than zinc. Copper is contained in many foods and is part of a healthy diet.

The earlier the System is used in the development of the cold or flu, the greater the chance of stopping the illness before it takes hold. The user gently rubs the tip of the wand for up to a minute on the mucous membranes that line the inner cavity of the nostril. The user may do so again in half an hour or so, and perhaps a third or fourth time at half hour intervals if the tickle in the nose or the scratchy throat returns or has not been completely eliminated.

In all examples, it is important that the user keep the Sanitizer System clean and free of dirt, oil, film, or other foreign matter. Copper is known to kill pathogens by direct physical contact of the copper surface with the individual bacterium or virus. Thus any foreign matter on the surface of the System may reduce its effectiveness. Though the System is designed based in part on results of studies of “Antimicrobial Copper”, the inventor at present does not apply the term “Antimicrobial Copper” to the System. The term is a trade name of the Copper Development Association, which, in conjunction with the EPA, sets requirements for its use.

In some embodiments, the invention features a method for alleviating a common cold in a mammal, including human, said method comprising placing a stem 120 comprising an exterior copper material into an inner cavity of a nostril of the mammal, wherein a distal end 121 of the stem is moved around in contact with a bottom floor of the inner cavity of the nostril swabbing the inner cavity of the nostril to deposit a residue of antimicrobial material thereon. The stem may or may not be attached to any base.

### Example 7

### Method of Application and Use

Directions for Use:

USE AS SOON AS POSSIBLE at the first sign of cold or flu. A delay may reduce any benefit of copper. A tickle in the nose or scratchy throat can be early signs of a cold. Some people only notice later signs, like a cough, congestion or sinus pressure, which may mean the cold has already started. Copper may still stop it, or at least make it shorter or less severe. The longer you wait, the less benefit is likely.

The first signs of flu may include aches, fever, chills, headache, weakness, fatigue, sore throat or dry cough. Copper can kill flu viruses and may help if applied early and repeated several times a day for 2-3 days. It is not a substitute for flu shots, however.

Cold and flu viruses can incubate for days before you notice signs. If you have been around people with the cold or flu you may already have the viruses in your nose. You may apply copper even before signs appear.

WASH CopperZap™ AND YOUR HANDS thoroughly with soap and warm water and rinse and dry just before each time you use it in your nose.

INSERT THE TIP VERY GENTLY into your nostril. Push gently into the inner cavity, BEYOND the outer opening where hair grows. The curve of the stem should point the tip down into the bottom of your nostril where viruses collect. Tilt the handle up so that tip gets down in the valley of the nostril. Push gently as far back in the nostril as it will reach without pain. If it hurts, STOP, that's too far.

SLIDE THE TIP VERY; GENTLY BACK AND FORTH for 60 seconds along the valley bottom of the nostril and along the valley sides near the bottom of the nostril. Dry off the tip with tissue and REPEAT IN THE OTHER NOSTRIL.

Putting something in your nose may make your eyes water or make you sneeze. If you sneeze, blow your nose and start over but only for about 30 seconds. Start over only once even if you sneeze again.

USE AGAIN AFTER 1 HOUR if signs of cold or flu remain or return. DO NOT USE more than 4 times in one day or more than 3 days in a row.

CLEAN BEFORE AND AFTER each use by washing CopperZap™ AND YOUR HANDS with soap and warm water.

RUB YOUR FINGERS AND THUMB ON THE HANDLE for 60 seconds if you may have been exposed to harmful germs but can't wash your hands right away. This may leave a slight residue of copper on your fingers. It may continue to protect for a short while against new germs arriving.

People exposed to high germ levels, such as workers in health care, day care, and elder care, may want to slide the top around in the outer nostril occasionally. Copper can help reduce the spread of infectious illness.

If you have just visited a hospital or doctor's office or been in public or handled money, rub the handle on your cheeks and around your nose. Copper may add protection due to its ability to kill germs.

Copper develops a natural patina or tarnish and changes color over time. The EPA says this does not reduce the power of copper to kill germs. If you prefer a shinier appearance, polish your CopperZap™ with a household metal polish or copper cleaner available in grocery stores. Follow directions, use rubber gloves, and WASH OFF ALL THE POLISH THOROUGHLY so it doesn't get in your nose.

Copper is a soft metal, so it is easily scratched or marred. This should not reduce its effectiveness.

USE ONLY AS DIRECTED. FOR ADULT USE ONLY. STEP 1. Insert very gently into nostril, with tip pointed downward. TILT TOP DOWN INTO BOTTOM OF INNER CAVITY OF NOSTRIL. STEP 2. Push tip slowly and gently as far back as you can without hurting and rub gently for 60 seconds all along the bottom and low part of the inside of the nostril. SLIDE IN AND OUT ALONG BOTTOM AND LOW AREAS INSIDE THE NOSTRIL. STEP 3. Withdraw slowly and gently, wipe off tip and stem with clean tissue or cloth, and repeat in other nostril.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method of killing or inhibiting pathogens of a mucosal area, said method comprising:
(a) locating a mucosal area;
(b) introducing a substrate to the mucosal area, the substrate comprising an antimicrobial metal; and
(c) using the substrate to directly contact the pathogens residing in the mucosal area, wherein the method is intended to kill or inhibit the pathogens in the mucosal area.

2. The method of claim 1, wherein the substrate comprises a non-microbial metal material either fully or partially coated with the antimicrobial metal.

3. The method of claim 1, wherein the antimicrobial metal is coated, lined, clad, or plated onto the substrate.

4. The method of claim 1, wherein the substrate comprises a combination of antimicrobial metal and non-antimicrobial metal.

5. The method of claim 1, wherein the antimicrobial metal comprises copper, zinc, silver, gold, an antimicrobial alloy, or a combination thereof.

6. The method of claim 5, wherein the antimicrobial alloy comprises brass, bronze, or a combination thereof.

7. The method of claim 1, wherein the substrate comprises a device adapted to be applied to the mucosal area.

8. The method of claim 1, wherein the step of directly contacting the pathogens residing in the mucosal area comprises sliding the substrate around an inner cavity of the nostril to contact the mucous or mucous membranes with the substrate.

9. The method of claim 1, wherein the step of directly contacting the pathogens residing in the mucosal area with the substrate deposits a trace amount of antimicrobial metal ions onto the mucosal area.

10. The method of claim 9, wherein the trace amount of antimicrobial metal helps to kill or inhibit the pathogens.

11. A method of killing or inhibiting pathogens of a vaginal area or anal area, said method comprising:
(a) locating a vaginal area or an anal area;
(b) introducing a substrate to the vaginal area or the anal area, the substrate comprising an antimicrobial metal;
(c) using the substrate to directly contact the pathogens residing in the vaginal area or the anal area, wherein the method is intended to kill or inhibit the pathogens in the vaginal area or anal area.

12. The method of claim 11, wherein the substrate comprises a non-microbial metal material either fully or partially coated with the antimicrobial metal.

13. The method of claim 11, wherein the antimicrobial metal is coated, lined, clad, or plated onto the substrate.

14. The method of claim 11, wherein the substrate comprises a combination of antimicrobial metal and non-antimicrobial metal.

15. The method of claim 11, wherein the antimicrobial metal comprises copper, zinc, silver, gold, an antimicrobial alloy, or a combination thereof.

16. The method of claim 15, wherein the antimicrobial alloy comprises brass, bronze, or a combination thereof.

17. The method of claim 11, wherein the substrate comprises a device adapted to be applied to the vaginal area or the anal area.

18. The method of claim 11, wherein the step of directly contacting the pathogens residing in the vaginal area or the anal area with the substrate comprises sliding the substrate around the vaginal area or anal area to contact the tissues with the substrate.

19. The method of claim 11, wherein the step of directly contacting the pathogens residing in the vaginal area or anal area with the substrate deposits a trace amount of antimicrobial metal ions onto the vaginal area or the anal area.

20. The method of claim 19, wherein the trace amount of antimicrobial metal helps to kill or inhibit the pathogens.
